(19) United States
(12) Plant Patent Application Publication
(10) Pub. No.: US 2012/0060249 P1
(43) Pub. Date: Mar. 8, 2012

(54) Carnation plant named 'CFPC Ohana'

(50) ;

(76) Inventor: Oscar Hasegawa, Salinas, CA (US)

(21) Appl. No.: 12/807,543

(22) Filed: Sep. 8, 2010

(51) Int. Cl. A01H 5/00

(52) U.S. Cl. PLT/281

(57) ABSTRACT

A new variety of carnation plant named ‘CFPC Ohana’, having a compact form of plant with well presented flowers which have a purple pink center and a flushed pinkish white border.

### LATIN NAME OF THE GENUS AND SPECIES

Botanical classification: Dianthus caryophyllus.

### VARIETY DENOMINATION

The new carnation variety denomination is ‘CFPC Ohana’.

### BACKGROUND OF THE INVENTION

The present invention comprises a new and distinct cultivar of hybrid carnation botanically known as Dianthus caryophyllus, and referred to by the cultivar name ‘CFPC Ohana’. The new variety, identified as selection no. 090485, originated from a cross made by Oscar Hasegawa in a controlled breeding program in Salinas, Calif.

The female parent of the new variety is known as selection no. 080184 and the male parent is known as selection no. 080224. The new variety ‘FPC Ohana’ has been asexually reproduced by vegetative side shoot cuttings in Salinas, Calif. and the distinguishing characteristics are retained through successive generations of asexual reproduction.

### BRIEF SUMMARY OF THE INVENTION

‘CFPC Ohana’ is a potted spray-type carnation plant variety having a compact form of plant with well presented flowers which have a purple pink center and flushed pinkish white borders.

### Comparison with Female Parent

Plants of the new carnation variety ‘CFPC Ohana’ are similar to the female parent selection no. 080184 in plant habit and growth rate. Under similar growing conditions in Salinas, Calif., under commercial practice, plants of the new carnation variety ‘CFPC Ohana’ differed from plants of the female parent in the following characteristic:
1. The new variety ‘CFPC Ohana’ produces flowers having a purple pink center and a flushed pinkish white border whereas the female parent selection no. 090184 produces purple flowers.

### Comparison with Male Parent

Plants of the new carnation variety ‘CFPC Ohana’ are similar to the male parent selection no. 080224 in plant habit and growth rate. Under similar growing conditions in Salinas, Calif., under commercial practice, plants of the new carnation variety ‘CFPC Ohana’ differed from plants of the male parent in the following characteristic:
1. The new variety ‘CFPC Ohana’ produces flowers having a purple pink center and a flushed pinkish white border whereas the male parent produces pink with white bicolor flowers.

### Comparison with other Similar Varieties

Plants of the new carnation variety ‘CFPC Ohana’ are similar to plants of the variety ‘CFPC Tess’ in plant habitat and growth rate. Under similar growing conditions, however, in Salinas, Calif., under commercial practice, plants of the new carnation variety ‘CFPC Ohana’ differed from plants of ‘CFPC Tess’ in the following characteristics:
1. The new variety ‘CFPC Ohana’ produces flowers having a purple pink center and a flushed pinkish white border whereas ‘CFPC Tess’ produces purple flowers.
2. Plants of the new variety ‘CFPC Ohana’ are shorter than plants of the variety ‘CFPC Tess’.

### BRIEF DESCRIPTION OF ILLUSTRATION

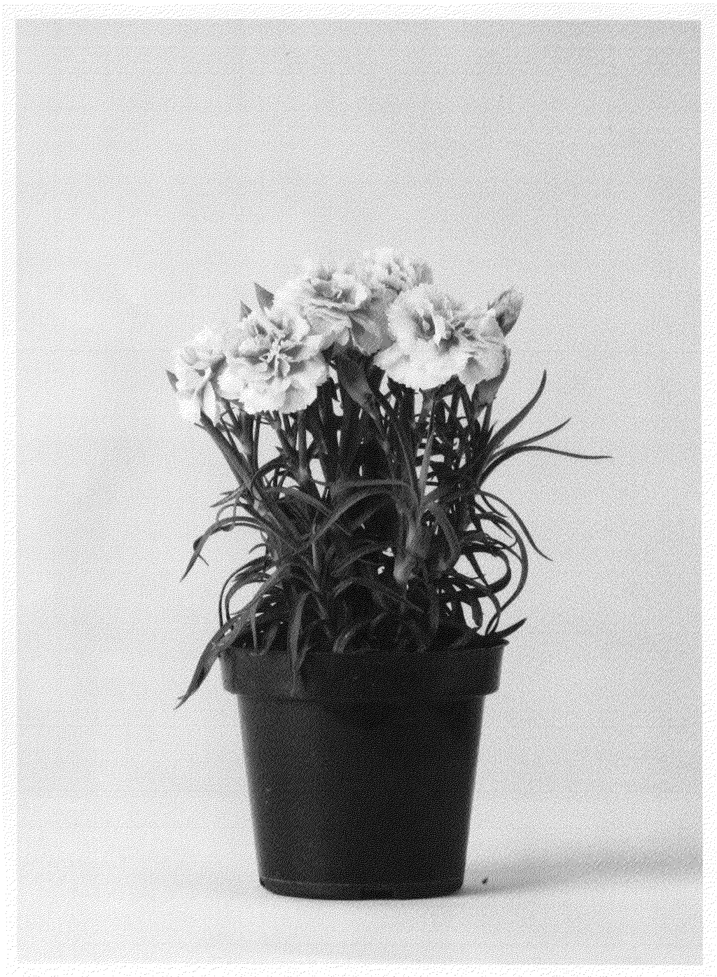

Typical specimens of the plant and flowers for the new carnation variety ‘CFPC Ohana’ are shown in the accompanying photograph. The colors shown are as true as possible within the usual limits of this kind of illustration. FIG. 1 is a whole plant view of the new variety ‘CFPC Ohana’ grown in a pot. The plant shown in the illustration is 139 days from date of planting.

### DETAILED BOTANICAL DESCRIPTION

The following description of the new carnation variety ‘CFPC Ohana’ is of plants 139 days from a planting date of Dec. 6, 2009 grown in a commercial greenhouse in Salinas, Calif. The cultivar has not been observed under all possible environmental conditions. The phenotype may vary significantly with variations in the environment such as temperature, length of day and light intensity without any variance in genotype.

Plants of the new variety have been grown successfully under temperature conditions averaging about 18° C. at night and about 24° C. to 28° C. during the day under light conditions of about 14 hours. Plants are daylight neutral and flower throughout the year. Chemical growth regulators are not required for production. The new variety may be produced as a potted spray-type carnation. To produce a commercial product the plants may be pinched once with the center bud removed. Pinched plants are about 6 inches tall and about 6 inches in diameter. Flower diameter averages about 2 inches with a crown of about 1 inch. Foliage is generally about 2¾ inches long and about ¼ inch at the widest point. The typical container size for commercial growth is 4½ inches.

It has been observed that the shelf life of the new variety is about 26 days with a time to first bud opening of about 15-16 weeks. The commercial classification of the new carnation variety is a potted spray-type. The plant hardiness zone has not been determined.

The following description is with respect to a plant produced as a potted spray-type carnation. The plant and flower measurements and foliage size are established as an average using standard commercial cultural practices. In the description of this new carnation variety, color values have been taken from The Royal Horticultural Society Colour Chart (R.H.S.C.C.).
Plant:
   Height (inches).—6.
   Form.—herbaceous.
   Growing habit.—semi upright.
   Branching characteristics.—3-4 laterals per stem.
   Breaking action.—6-7 breaks per plant with strong straight stems.
   First appearance of color in buds.—105 days after planting.
   Vigor.—excellent.
   Shelf life.—26 days.
   Blooming habit.—spray type.
   Blooming season.—year round.
   Lastingness of blooms.—about 14 days.
   Disease (susceptibility/resistance observed).—excellent resistance to soil-borne diseases such as Fusarium oxysporum and F. graminearium, and root rots such as damping off, water molds and similar types, as are typical of carnations. Pest susceptibility/resistance not evaluated or observed Drought and temperature susceptibility/resistance not evaluated or observed.
   Mutation prone.—no.
   Propagation.—Type: side shoot cuttings Time to rooting: 18 days with soil temperatures of about 18° C. to 21° C. Rooting habit: good fibrous system.
   Growth treatments or special conditions.—single pinch.
Foliage:
   Stem.—Length (inches): 4 Diameter (inches): ¼ (at base) Texture: smooth Cross section: edged Color: 141 A internode 143 D node.
   Number of leaves per lateral branch.—22.
   Arrangement of leaves.—bi-lateral along the stem Shape of Leaf: long, narrow, lanceolate Size of Leaf: Width (inches): ¼ Length (inches): 3 Leaf Apex: acute Edge: smooth Base: smooth Aspect: elliptie Surface texture: Top: smooth Bottom: smooth.
   Leaf venation.—prominent mid-vein at underside.
   Leaf longitudinal axis.—recurbed.
   Leaf cross section (upper side).—weakly concave.
   Leaf color.—Mature Leaf, upper side: 137 A; under side: 137 B Young Leaf, upper side: 137 D; under side: 138 A.
   Leaf waxy layer.—medium.
Flower:
   Flower appearance.—matte to shiny.
   Flower type.—single/double.
   Flower profile of upper part of corolla.—convex.
   Flower profile of lower part of corolla.—flat convex.
   Flower shape.—somewhat domed shape rounded.
   Number of blossoms per branch.—5 Depth of fully expanded blossoms (inches): 1 Diameter of fully expanded blossoms (inches): 2.
   Calyx.—Sepals number: 5 Length (inches): 1 Width (inches): 7/16 Color: outer side base: 145 C outer side middle to tip 144 A inner side: 145 D Texture: smooth Appearance: matte with waxy bloom Shape: cylindrical.
   Flower petals.—Form/Shape: fan shape Petal Edge: serrated: width: 1/16 inch depth: 1/16 inch edge type: crenate-dentate Texture: smooth Appearance: matte-shiny Petal surface of blade: undulating Number per flower: 36 Outer petals: Length (inches): 1¾ Width (inches): 1 Inner petals: Length (inches): 1¼ Width (inches): 7/16.
   Fragrance.—absent.
   Flower bud (at onset of color).—Length (inches): 1 Diameter (inches): 7/16 Form/Shape: cylindrical.
   Flower color.—1. General tonality: Pink 2. Petals, upper surface: 67 C 3. Petals, lower surface: 155 C 4. Petals, edge: 69 D 5 Petals, center: 67 C 6. Early Bud: 67 D.
   Color distribution.—flushed.
   Petal macule (random spots).—absent.
   Flower progression with age.—color becomes very slightly to lighter color.
Reproductive organs:
   Gynoecium.—Pistillate Pistil number: 1 Pistil length (inches): 1 Stigma color: 65A Stigma shape: filamentous Style color: 155 D-white Style length (inches): 1 Style number: two.
   Ovary.—Shape: ovoid Surface: smooth Color: Base: 145 C Body: 145 B Apex: 145 A.
   Androecium.—none produced.
   Fruit and seeds.—none produced.

What is claimed is:

1. A new and distinct variety of carnation plant, substantially as described and illustrated herein.
